(19) United States
(12) Patent Application Publication
(10) Pub. No.: US 2012/0029481 A1
(43) Pub. Date: Feb. 2, 2012

(54) CONNECTION PIECE FOR THE TUBE OF AN ENTERAL FEEDING TUBE AND ASSEMBLY OF AN ENTERAL FEEDING TUBE AND AN ENTERAL TRANSFER SYSTEM

(75) Inventors: Bernhard Pech, Hunstetten (DE); Helmut Schäfer, Usingen (DE)

(73) Assignee: V. KRÜTTEN MEDIZINISCHE EINMALGERÄTE GmbH, Idstein (DE)

(21) Appl. No.: 13/258,534

(22) PCT Filed: Apr. 3, 2010
(86) PCT No.: PCT/EP10/02143

§ 371 Date: Sep. 22, 2011

(30) Foreign Application Priority Data
Apr. 7, 2009 (DE) 10 2009 016 373.5

(51) Int. Cl. A61M 39/10

(52) U.S. Cl. 604/533

(57) ABSTRACT

The invention relates to a connection piece (4) for the tube (3) of an enteral feeding tube (1), having a proximal fitting piece (4A) for fitting the tube of an enteral feeding tube, and a distal fitting piece (4B) for fitting a connection piece (8) of an enteral transfer system (2). The connection piece (4) according to the invention of the feeding tube (1) comprises two rigidly connected sections (9), (10), of which the first section (9) is made of a material which has a higher hardness than the material which the second section (10) of the connection piece is made of. The two-piece structure of the connection piece made of the two materials of different hardness has the advantage that the material properties of the two sections (9), (10) can be adapted to the different requirements for the respective connections.

The invention relates to a connection piece for the tube part of an enteral feeding tube, having a proximal attachment piece for the attachment of the tube part of an enteral feeding tube, and a distal attachment piece for the attachment of a connection piece of an enteral transfer system. In addition, the invention relates to an enteral feeding tube with a tube part to which the connection piece is attached. The invention further relates to an assembly composed of an enteral feeding tube and of an enteral transfer system.

In enteral nutrition, a liquid nutrient solution is administered to the patient using an enteral feeding tube. The enteral nutrient solution is made available in a container, which is connected to the enteral feeding tube by way of what is called a transfer system. The enteral transfer system comprises a tube line with a proximal connection piece and a distal connection piece. The proximal connection piece is connected to the feeding tube, and the distal connection piece is connected to the nutrient solution container, such that the nutrient solution flows from the container into the feeding tube by way of the tube line of the transfer system.

The nutrient solution container is generally replaced several times a day, whereas it is recommended that the transfer system be replaced every 24 hours. The feeding tube can be used for up to several months.

To connect the connection tube of the transfer system to the tube part of the feeding tube, the known Luer lock connectors are used in the prior art, such that transfer system and feeding tube can be connected to each other without the danger of the tube part of the feeding tube accidentally detaching from the connection tube of the transfer system during the administration of the nutrient solution. However, a disadvantage is that the known Luer lock connections used in enteral administration may pose a risk to patients in the sense that the Luer lock connections used in enteral administration may be confused with Luer lock connections that are used particularly in intravenous administration and in ventilation techniques. For example, if enteral nutrition were to be administered accidentally through an intravenous Luer lock connection, there would be a considerable risk to the patient.

Improved safety is achieved in the prior art by the fact that the enteral transfer system has a female Luer lock connection piece, while the enteral feeding tube has a male Luer lock connection piece. However, since intravenous tubes have a female Luer lock connection piece, this rules out the possibility of an enteral transfer system being connected directly to the intravenous tube. However, because of the large number of available Luer lock adapters offered by various manufacturers, mix-ups cannot be completely ruled out.

In addition to the transfer systems and feeding tubes with Luer lock connectors, feeding tubes are known that have a funnel-shaped connection piece. The various manufacturers offer feeding tubes with attachment funnels of different diameter. To be able to produce a connection to the attachment funnels of the known feeding tubes, the prior art discloses step-shaped adapters, which are generally connected releasably to the connection tube of the transfer system via a Luer lock connection.

The known stepped adapters have the advantage that a connection to attachment funnels of feeding tubes of different diameter is possible. The stepped adapter is plugged into the attachment funnel and, depending on the diameter of the attachment funnel, that portion of the stepped adapter whose diameter corresponds to the diameter of the attachment funnel comes into contact with the attachment funnel. The two parts are in this case fixed by the frictional force of the jacket surfaces of stepped adapter and attachment funnel.

A disadvantage is that the retention force between stepped adapter and attachment funnel may not be sufficient. In particular when using fat-containing liquid feeds, there is the risk of the connection accidentally coming loose. Aging effects, which influence the material properties of the attachment funnel and the geometry of the latter, may also lead to a reduced retention force.

Screw connections, which are also used in the field of enteral nutrition, are known from WO 2005/055919 A1. A disadvantage is that the known screw connections cannot be used universally and instead are limited to the particular feeding tubes or transfer systems having suitable connections.

WO 2008/049568 A1 describes a connection piece having an attachment piece for the attachment of a connection piece of the tube part of an enteral feeding tube, and having an attachment piece for the attachment of the connection tube of a transfer system. The attachment piece for the connection piece of the tube part of the enteral feeding tube has a locking element, which is designed in such a way that, on the one hand, it is possible to produce a releasable form-fit connection to a connection piece of a tube part of an enteral feeding tube that has a locking element complementing the locking element of the attachment piece, and, on the other hand, it is possible also to produce a force-fit but not a form-fit releasable connection to connection pieces of the tube parts of other feeding tubes that do not have such a locking element. The connection piece is a one-piece injection-molded part that can be produced in large batch numbers.

It is an object of the invention to make available a connection piece for an enteral feeding tube, which connection piece can be used universally and is easy to handle and permits a safe but releasable connection of transfer system and feeding tube. Another object of the invention is to create an enteral feeding tube that can be used universally and is easy to handle and that can be safely connected to a transfer system without the danger of accidental loosening of the connection. In addition, it is an object of the invention to make available an assembly composed of a feeding tube and of a transfer system, which assembly can be used universally and is easy to handle and in which feeding tube and transfer system can be safely connected to each other.

According to the invention, these objects are achieved by the features set forth in claims 1, 18 and 19. Advantageous embodiments form the subject matter of the dependent claims.

The connection piece according to the invention for an enteral transfer system has a proximal attachment piece for the attachment of a connection piece of a tube part of an enteral feeding tube, and a distal attachment piece for the attachment of the connection tube of a transfer system.

The connection piece according to the invention is composed of two sections firmly connected to each other, of which the first section is made of a material that is harder than the material from which the second section of the connection piece is made. The two-part construction of the connection piece from the two materials of different hardness has the advantage that the material properties of the two sections can be adapted to the different requirements of the respective connections. For example, the section made of the harder material can be used to create a preferably form-fit connection and the section made of the softer material can be used to create a preferably force-fit connection between the connection piece according to the invention and the connection piece of the connection tube of a transfer system, the form-fit connection being intended to be unyielding and the force-fit connection being intended to be yielding. The two sections made of materials of different hardness can be firmly connected to each other by welding or adhesive bonding, for example. Moreover, the connection piece with the two sections can be produced inexpensively in large batch sizes as a multi-component injection-molded part.

It is advantageous that the connection piece according to the invention avoids confusion with Luer lock connections used in non-enteral access routes, because of the incompatibility of the connection pieces taking part in the connection. In a preferred embodiment of the connection piece, at least part of the first section of the connection piece, made of a harder material than the second section of the connection piece, is designed as a locking element, with which it is possible to produce a releasable form-fit connection to a connection piece of a transfer system that has a locking element complementing the locking element of the feeding tube. The harder material of the first section ensures that the locking element has sufficient strength to guarantee a form-fit connection that has a high degree of safety. The harder material also has the advantage that the locking element is subjected to only minimal wear by abrasion.

The first section of the connection piece is preferably made of a material chosen from the group comprising rigid polyurethane, rigid PVC, ABS and polycarbonate, and compounds of these materials with other materials, for example plasticizers, etc. The material from which the second section of the connection piece is made is preferably a material chosen from the group comprising soft polyurethane, soft PVC and silicone, and compounds with other materials, for example plasticizers, etc.

The material of the second section preferably has a Shore hardness of between Shore A 90 and Shore A 30, with the Shore hardness being determined according to DIN 53505 and DIN 7868.

In another preferred embodiment, the locking element, with which the connection piece of the connection tube of an enteral transfer system is secured on the connection piece of the tube part of an enteral feeding tube, is designed as a rotary locking element (rotary closure), preferably as a screw connection element with a thread or as a bayonet connection element, such that the connection piece of the enteral transfer system can be easily and securely connected to the connection piece of the enteral feeding tube. The important point is simply that the connection is a releasable form-fit connection. In another preferred embodiment, at least part of the second section of the connection piece, made of a softer material, is designed as a hollow attachment piece, with which it is possible to produce a releasable force-fit connection to a connection piece of the connection tube of an enteral transfer system. The softer, more elastic material has the advantage that a secure force-fit connection can be produced even when there is not an optimal register of the respective connection pieces, since the softer material can better adapt to the individual geometries of different connection pieces.

The hollow attachment piece of the connection piece preferably has an inwardly narrowing receiving channel into which the connection piece of the connection tube of an enteral transfer system can be inserted with a snug fit.

In another preferred embodiment of the connection piece, the first or second section of the connection piece has a part designed as a conical receiving piece into which the connection piece of the connection tube of an enteral transfer system can be inserted with a snug fit. The conical receiving piece ensures a reliable seal, but it does not create a form-fit connection like the part of the connection piece designed as locking element.

In another preferred embodiment of the connection piece, the first or second section of the connection piece has a part designed as a cylindrical fastening piece on which the tube part of an enteral feeding tube is fastened.

The conical receiving piece for the connection piece of the connection tube of an enteral transfer system and the cylindrical fastening piece for the tube part of an enteral feeding tube can in principle be a component of the first or second section. In the case where the conical receiving piece is a component of the first section of the connection piece made of a harder material, the first and second sections of the connection piece are preferably designed as hollow bodies that are firmly connected to each other at the ends. In this embodiment, it is not only the locking element that is made of a harder material but also the conical receiving piece and the cylindrical fastening piece. In the alternative embodiment in which the conical receiving piece and the cylindrical fastening piece are a component of the second section made of a softer material, the first and second sections of the connection piece are preferably designed as hollow bodies, with the first section being inserted into the second section. In this embodiment, only the locking element is made of the harder material, while all the other parts of the connection piece are made of the softer material.

The connection piece preferably has a closure cap which is used to close the distal attachment piece and which, by way of a tab, is secured permanently to the second section of the connection piece, said closure cap and tab being made of the same material as the second section of the connection piece. The closure cap with the tab is thus made of a softer material that has sufficient flexibility.

In a particularly preferred embodiment, the closure cap is composed of a lid part, with an opening for the attachment of a syringe, and of a closure part which is used to close the opening in the lid part and is secured permanently to the lid part by way of a tab. After the closure cap fitted on the distal attachment piece has been opened, it is thus possible to inject a liquid, in particular a medicament, after which the injection opening can again be tightly closed. To inject a liquid, in particular a medicament, the connection piece in an alternative embodiment is provided with an additional attachment funnel that can be closed by a closure cap.

The enteral feeding tube according to the invention has the connection piece according to the invention, which connection piece is attached to the tube part, while the assembly according to the invention comprises the enteral feeding tube, to the tube part of which the connection piece is attached, and an enteral transfer system, to the connection tube of which a connection piece is attached that can be connected to the connection piece of the enteral feeding tube.

Various illustrative embodiments of the invention are explained in more detail below with reference to the drawings, in which:

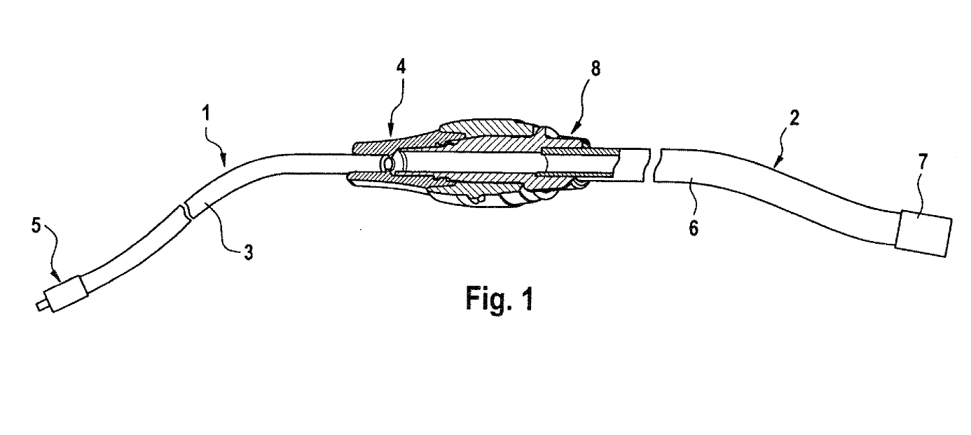

FIG. 1 shows an assembly according to the invention composed of an enteral feeding tube and of an enteral transfer system,

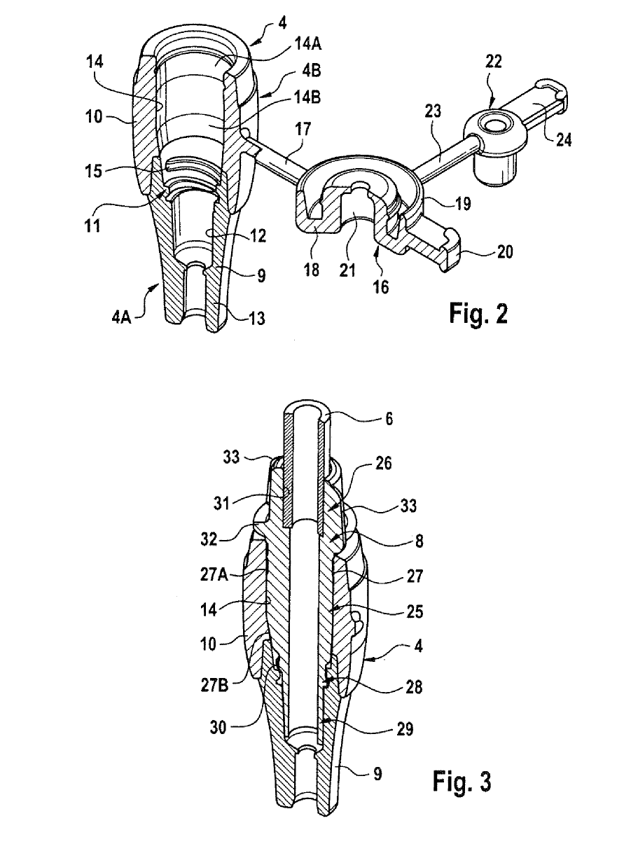

FIG. 2 shows the connection piece of the enteral feeding tube in a sectioned perspective view,

FIG. 3 shows a cross section through the inter-connected connection pieces of the feeding tube and of the transfer system,

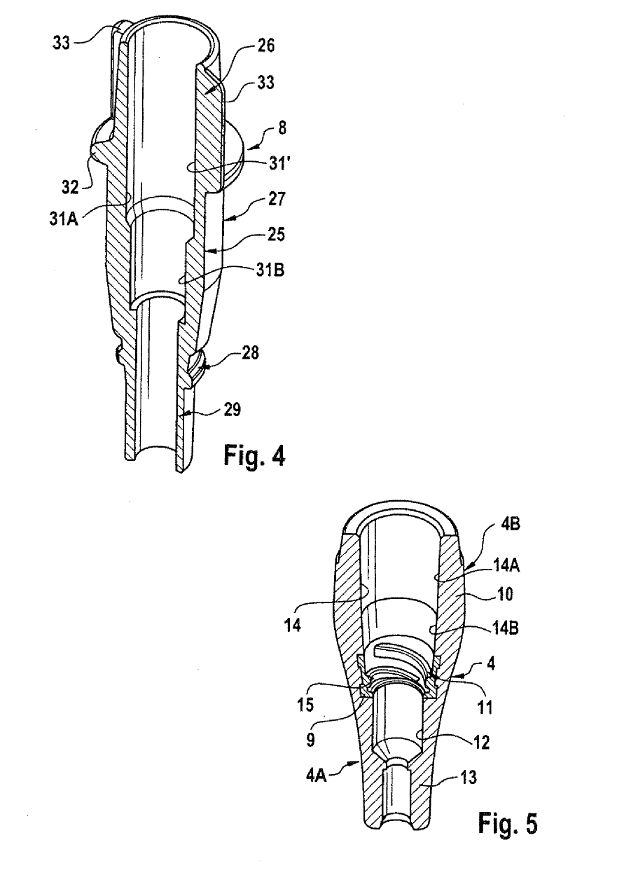

FIG. 4 shows the connection piece of the enteral transfer system in a partially sectioned perspective view,

FIG. 5 shows an alternative embodiment of the connection piece according to the invention of the enteral feeding tube in a partially sectioned perspective view,

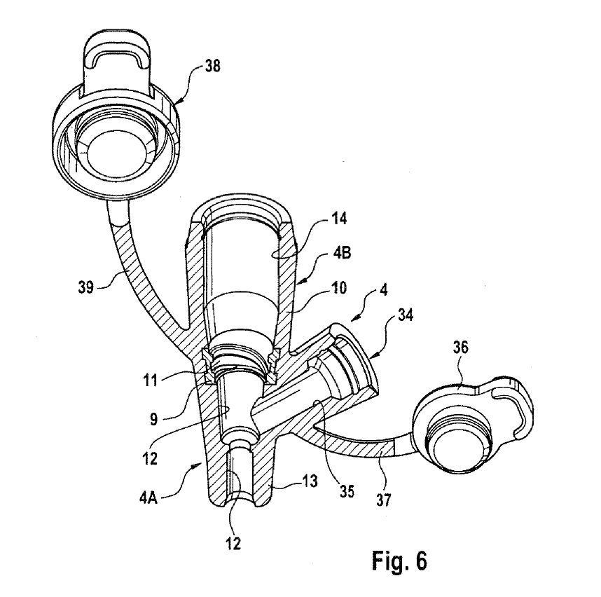

FIG. 6 shows an alternative embodiment of the connection piece of the enteral transfer system in a partially sectioned perspective view, and

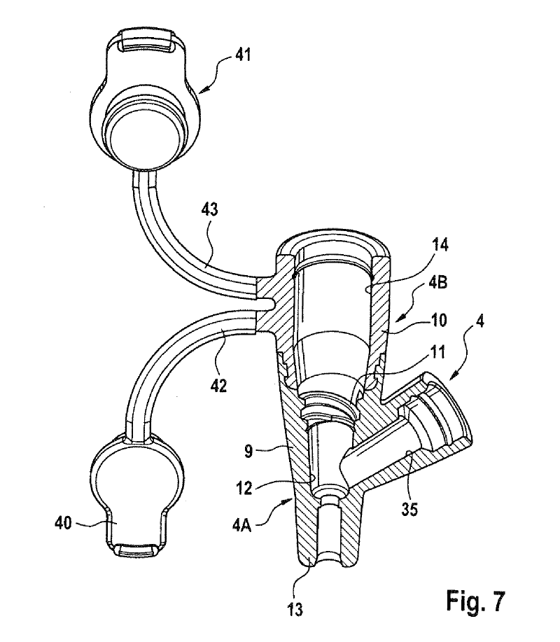

FIG. 7 shows another alternative embodiment of the connection piece according to the invention of the enteral feeding tube in a partially sectioned perspective view.

FIG. 1 shows an assembly according to the invention composed of an enteral feeding tube 1 and of an enteral transfer system 2, which are connected to each other in order to administer an enteral nutrient solution from a nutrient solution container (not shown) to a patient. Since enteral transfer systems and feeding tubes as such are known to persons skilled in the art, the description is by and large confined to the parts that are relevant to the invention.

The feeding tube 1 has a tube part 3 with a distal connection piece 4 at the distal end of the tube, and, at the proximal end, it has a proximal endpiece 5 that is to be introduced into the patient.

The transfer system 2 has a connection tube 6 with a distal connection piece 7 at the distal end of the tube and with a proximal connection piece 8 at the proximal end of the tube. The distal connection piece 7 serves to attach the transfer system to a nutrient solution bag (not shown), while the proximal connection piece 8 serves for the attachment of the feeding tube 1. For this purpose, the distal connection piece 4 of the feeding tube 1 is connected to the proximal connection piece 8 of the transfer system 2.

Various illustrative embodiments of the connection pieces of feeding tube 1 and transfer system 2 are described in detail below.

FIG. 2 shows a first illustrative embodiment of the connection piece 4 according to the invention, to which the distal end of the tube part 3 of the feeding tube 1 is attached. The connection piece 4 of the feeding tube 1 has a proximal attachment piece 4A for the tube part of the feeding tube, and a distal attachment piece 4B for the connection piece of the connection tube of the transfer system. The connection piece 4 is composed of a first section 9 and of a second section 10, the two sections 9, 10 being firmly connected to each other. The first section 9 of the connection piece 4 is made of a material that is harder than the material from which the second section 10 is made. The first section 9 is made, for example, of rigid polyurethane, rigid PVC, ABS (acrylonitrile/butadiene/styrene copolymer) or polycarbonate, or compounds of these materials with other materials, for example plasticizers. The second section 10 of the connection piece 4 is made, for example, of soft polyurethane, soft PVC or silicone, or compounds of these materials with other materials, for example plasticizers. The second section 10 of the connection piece has a Shore hardness of between Shore A 90 and Shore A 30, with the Shore hardness being determined according to DIN 53505 and DIN 7868.

In the illustrative embodiment in FIG. 1, part of the first section 9, which is made of a harder material than the second section 10, is designed as a locking element 11. With the locking element 11, it is possible to produce a releasable form-fit connection to the proximal connection piece 8 of the transfer system 2, which has a locking element complementing the locking element 11 of the connection piece 4 of the feeding tube 1. The proximal connection piece 8 of the transfer system 1 is described in more detail below.

In addition to the locking element 11, the first section 9 of the connection piece 4 has a conical receiving piece 12, in which a portion of the connection piece 8 of the transfer system 2 is received with a snug fit. The proximal attachment piece 4A of the connection piece 8 is a cylindrical fastening piece 13 into which the tube part 3 of the feeding tube 1 is pushed. The tube part 3 of the feeding tube 1 can be adhesively bonded or welded to the cylindrical fastening piece 13 of the connection piece 4. The fastening piece 13 is a component part of the first section 9 of the connection piece 4.

In the illustrative embodiment in FIG. 1, in which only a part of the first section 9 is designed as locking element 11, the first and second sections 9 and 10 of the connection piece 4 are hollow bodies that are made of different materials and that are firmly connected to each other at their ends, for example welded or adhesively bonded. An outward-lying shoulder of the second section 10 engages over an inward-lying shoulder of the first section 9. The connection piece 4 can also be produced as a multi-component injection-molded part.

At the upper portion shown in FIG. 1, the hollow body of the first section 9 of the connection piece 4 has as locking element a screw thread 15, which is an internal thread. The internal thread 15 is adjoined, in the middle portion of the hollow body, by the conical receiving piece 12 for the connection piece 8 of the transfer system 2. The conical receiving piece 12 merges into the cylindrical fastening piece 13 at the lower portion of the hollow body.

The second section 10 of the connection piece 4 is made of a softer material than the first section 9 and has a receiving channel 14 into which the connection piece 8 of the transfer system 1 can be inserted with a snug fit. The receiving channel 14 of the second section 10 narrows in the direction of the first section 9 of the connection piece 4. To permit an improved register and thus an improved force-fit engagement, the receiving channel 14 has an outwardly lying conical portion 14A, which is adjoined by an inwardly lying conical portion 14B. In this way, the first section 10 of the connection piece 4 acquires the form of a funnel-shaped attachment piece 15 for the connection piece 8 of the transfer system 2.

The connection piece 4 has a closure cap 16 with which the distal attachment piece 4B can be sealingly closed. The closure cap 16 is secured permanently, by way of a tab 17, to the second section 10 of the connection piece 4. Closure cap 16 and tab 17 are made of the same softer material as the second section 10 of the connection piece 4, such that both the distal attachment piece 4B and also the closure cap 16 with the tab 17 have sufficient flexibility. Since the locking element 11, by contrast, is made of a harder material, it is possible to produce a lasting, wear-resistant and firm connection.

The closure cap 16 has a lid part 18 and an edge part 19 onto which a short tab 20 is integrally formed, with which the closure cap 16 can be easily pulled off from the distal attachment piece 4B of the connection piece 4.

In the lid part 18 of the closure cap 16 there is a central opening 21 for the attachment of a syringe (not shown) with which a liquid, in particular a medicament, can be injected. The opening 21 of the closure cap 16 is preferably conical in order to be able to receive the conical tip of the syringe.

In order to close the opening 21 in the closure cap 16, a matching closure part 22 is provided, which is integrally formed permanently on the closure cap 16 by way of a tab 23. Also formed integrally on the closure part 22, there is a short tab 24 with which the closure part 22 can be easily pulled out of the opening 21 in the closure cap 16. The closure cap 16 with the two tabs 23 and 24 is again made of the same softer material as the distal attachment piece 4B of the connection piece 4.

FIG. 3 shows the proximal connection piece 8 of the enteral transfer system 2, which is firmly screwed onto the distal connection piece 4 of the enteral feeding tube 1. The proximal connection piece 8 of the transfer system 1 is designed as an elongate hollow body that has a proximal section 25 and a distal section 26. The proximal section 25 has an upper portion 27, which is adjoined by a middle portion 28 that merges into a lower portion 29. To permit an improved register and thus an improved force-fit engagement, the upper portion 27 of the proximal section 25 has an upper cylindrical portion 27A, which is adjoined by a downwardly narrowing lower conical portion 27B. The middle portion 28 is designed as a locking element 30, which complements the locking element 11 of the distal connection piece 4 of the feeding tube 1. In the present illustrative embodiment, the locking element is an external thread 30, which can be screwed into the internal thread 15 of the connection piece 4 of the feeding tube. The lower cylindrical portion 29 of the proximal section 25 can be inserted with a snug fit into the conical receiving piece 12 of the connection piece 4 of the feeding tube 1, such that both parts provide a seal relative to each other. This ensures that the connection piece 8 of the transfer system 2 can be connected in a leaktight manner to the connection piece 4 of the feeding tube 1.

FIG. 3 shows how the connection piece 8 of the transfer system 2 sits in the connection piece 4 of the feeding tube 1. The upper portion 27A of the proximal section sits in the distal attachment piece 4B of the connection piece 4, while the lower portion 28 of the proximal section 25 sits in the conical receiving piece of the connection piece 4. The seal can thus be provided not only on the sealing surface between the lower cylindrical portion 29 of the proximal section 25 and the conical receiving piece 12, but also on the sealing surface between the upper portion 27A of the proximal section 25 and the wall of the receiving channel 14 of the connection piece 8. Since the connection piece in the area of this sealing surface is made of a softer, more yielding material, a reliable seal is ensured. In principle, a seal can also be provided via the screw connection.

The distal section 26 of the proximal connection piece 8 has a cylindrical channel 31 into which the proximal end of the connection tube 6 of the transfer system is pushed with a snug fit. The connection tube 6 can be adhesively bonded or welded to the distal section 26. With a circumferential shoulder 32, the distal section 26 of the connection piece 8 of the transfer system 2 bears on the upper edge of the distal attachment piece 4B of the connection piece 4 of the feeding tube 1 when both connection pieces 4, 8 are firmly screwed onto each other. In order to be able to better grip the connection piece 8 of the transfer system 2 with the fingers, two radially protruding shoulders 33 are integrally formed on the distal section 26.

It is clear that a releasable form-fit connection can be produced using the two connection pieces 4, 8. The particular design of the connection piece 4 of the feeding tube 1 not only permits production of a connection to the particular connection piece 8 of the transfer system 1, but also to other connection pieces of differing design, such that the connection piece can be used universally. Attachment of the known stepped adapters is also possible. This is achieved by the fact that the connection piece 4 of the feeding tube 1 comprises the funnel-shaped distal attachment piece 4B which, while not permitting a form-fit connection, does permit a force-fit connection to other connection pieces or to the known stepped adapters. Since the attachment piece is made of a soft material that has sufficient flexibility, the attachment piece can adapt to the different diameters of the other connection pieces. It is assumed here that, because of the inwardly narrowing design of the receiving channel 14 of the attachment piece 4B, a sealing surface is created at least in one portion of the receiving channel.

The cylindrical lower portion 29 of the proximal section 25 of the connection piece 8 of the transfer system 2 has a smaller wall thickness than the upper and middle portions 27 and 28. The wall thickness of the lower portion 29 is dimensioned such that the connection piece can deform at this portion. When the connection piece 8 of the transfer system 1 is connected to a corresponding connection piece 4 of a feeding tube 1, the lower portion 29 of the connection piece 8 deforms in such a way that this portion “digs itself” into the connection piece of the feeding tube. A deformation of this kind takes place particularly if the connection piece 8 of the transfer system 2 is turned slightly upon insertion into the connection piece 4 of the feeding tube 1.

FIG. 4 shows an alternative embodiment of the proximal connection piece 8 of the enteral transfer system 2. The connection piece differs from the connection piece described with reference to FIG. 3 only in that the cylindrical channel 31′ for receiving the connection tube of the transfer system extends as far as the middle portion 28 of the proximal section 25 of the connection piece 8. In addition, cylindrical channel 31 is divided into two portions 31A and 31B of different diameter, with the diameter decreasing as far as the middle portion 28. The parts corresponding to one another are therefore provided with the same reference signs.

FIG. 5 shows another embodiment of the distal connection piece 4 of the enteral feeding tube 1. The illustrative embodiment in FIG. 5 differs from the embodiment described with reference to FIGS. 1 to 3 in that the locking element 11 is provided not just by part of the first section 9 but instead by the whole first section, which is made of a harder material than the second section 10 of the connection piece 4. The parts corresponding to one another are again provided with the same reference signs. Both sections 9, 10 are once again hollow bodies, but the first section 9 is inserted with a snug fit into the second section 10. Consequently, only the part of the connection piece with the internal thread 15 is made of the harder material. By contrast, the conical receiving piece 12 and the cylindrical fastening piece 13 are, like the distal attachment piece 4B, made of the softer material.

FIG. 6 shows another embodiment of the connection piece 4 of the feeding tube 1. The embodiment in FIG. 6 differs from the illustrative embodiment described with reference to FIG. 5 in that the connection piece 4 has an additional attachment funnel 34, which is formed integrally on the first section 9 of the connection piece 4. The attachment funnel 34 is a funnel-shaped attachment with a conical opening 35 into which the tip of a syringe for injecting a medicament can be inserted with a snug fit. The attachment funnel 34 can be closed with a closure cap 36, which is connected by a tab 37 to the attachment funnel. The attachment funnel and the closure cap and the tab are again made of the same softer material as the second section 10 of the connection piece 4. In addition, the embodiment in FIG. 6 has a closure cap 38 for closing the distal attachment piece 15. However, in contrast to the closure cap in the illustrative embodiment described with reference to FIGS. 1 to 3, the closure cap 39 does not have an opening for injection of a liquid. The parts corresponding to one another are again provided with the same reference signs.

FIG. 7 shows another embodiment of the connection piece 4 of the feeding tube 1. The parts corresponding to one another are again provided with the same reference signs. The connection piece in FIG. 7 differs from the connection piece in FIG. 6 in that both the locking element 11 and also the conical receiving piece 12 and the cylindrical fastening piece 13 are made of the harder material, while the distal attachment piece 4B is made of the softer material. To this extent, the structure of the connection piece in FIG. 7 corresponds to the connection piece in FIGS. 1 to 3. The two closure caps 40 and 41 for closing the attachment funnel 34 and the attachment piece 4B, respectively, are secured permanently to the connection piece 4 by way of tabs 42, 43, which are formed integrally on the distal attachment piece 4B of the connection piece.

1. A connection piece for the tube part of an enteral feeding tube, having a proximal attachment piece for the attachment of the tube part of an enteral feeding tube, and a distal attachment piece for the attachment of a connection piece of an enteral transfer system,
wherein,
the connection piece is composed of two sections firmly connected to each other, of which the first section is made of a material that is harder than the material from which the second section of the connection piece is made.

2. The connection piece as claimed in claim 1, wherein at least part of the first section of the connection piece is designed as a locking element with which it is possible to produce a releasable form-fit connection to a connection piece of a transfer system that has a locking element complementing the locking element of the connection piece.

3. The connection piece as claimed in claim 1, wherein or the material from which the first section of the connection piece is made is a material chosen from the group comprising rigid polyurethane, rigid PVC, ABS and polycarbonate.

4. The connection piece as claimed in of claim 1, wherein the material from which the second section of the connection piece is made is a material chosen from the group comprising soft polyurethane, soft PVC and silicone.

5. The connection piece as claimed in claim 1, wherein material from which the second section of the connection piece is made has a Shore hardness of between Shore A 90 and Shore A 30.

6. The connection piece as claimed in claim 1, wherein the locking element is designed as a rotary locking element, preferably a screw connection element.

7. The connection piece as claimed in claim 1, wherein at least part of the second section of the connection piece is designed as a hollow attachment piece, with which it is possible to produce a releasable force-fit connection to a connection piece of a transfer system.

8. The connection piece as claimed in claim 1, wherein the hollow attachment piece has an inwardly narrowing receiving channel into which a connection piece of a transfer system can be inserted with a snug fit.

9. The connection piece as claimed in claim 1, wherein the first section of the connection piece has a part designed as a conical receiving piece for the connection piece of a transfer system.

10. The connection piece as claimed in claim 1, characterized in that the first section of the connection piece has a part designed as a cylindrical fastening piece for a tube part of an enteral feeding tube.

11. The connection piece as claimed in claim 9, wherein the first and second sections of the connection piece are designed as hollow bodies that are firmly connected to each other at the ends.

12. The connection piece as claimed in claim 1, wherein the second section of the connection piece has a part designed as a conical receiving piece for the connection piece of a transfer system.

13. The connection piece as claimed in claim 1, wherein the second section of the connection piece has a part designed as a cylindrical fastening piece for a tube part of an enteral feeding tube.

14. The connection piece as claimed in claim 12, wherein the first and second sections of the connection piece are designed as hollow bodies, with the first section being inserted into the second section.

15. The connection piece as claimed in claim 1, wherein the connection piece has a closure cap which is used to close the distal attachment piece and which, by way of a tab, is secured permanently to the second section of the connection piece, said closure cap and tab being made of the same material as the second section of the connection piece.

16. The connection piece as claimed in claim 15, wherein the closure cap is composed of a lid part, with an opening for the attachment of a syringe, and of a closure part which is used to close the opening in the lid part and is secured permanently to the lid part by way of a tab.

17. The connection piece as claimed in claim 1, wherein the connection piece has an attachment funnel that can be closed by a closure cap.

18. An enteral feeding tube with a tube part, which has a proximal end and a distal end, and with a connection piece as claimed in claim 1, the distal end of the tube part of the enteral feeding tube being attached to the proximal attachment piece of the connection piece.

19. An assembly composed of an enteral feeding tube as claimed in claim 18 and of a transfer system that has a connection tube with a distal end and a proximal end, the proximal end of the connection tube being connected to a connection piece that can be connected to the distal attachment piece of the connection piece of the enteral feeding tube.

20. The assembly as claimed in claim 19, wherein the connection piece of the connection tube of the transfer system has a locking element, which is designed complementing the locking element of the connection piece of the tube part of the enteral feeding tube.
